(19) United States
(12) Patent Application Publication
(10) Pub. No.: US 2024/0180738 A1
(43) Pub. Date: Jun. 6, 2024

(54) DUAL USE DRAINAGE TUBE ASSEMBLY

(71) Applicant: BioDerm, Inc., Largo, FL (US)

(72) Inventors: Andrew P. Muser, Clarkesville, GA (US); Daniel Riveros, Redington Shores, FL (US); Terry Boersma, Scottsdale, AZ (US)

(73) Assignee: BioDerm, Inc., Largo, FL (US)

(21) Appl. No.: 18/526,854

(22) Filed: Dec. 1, 2023

Related U.S. Application Data
(60) Provisional application No. 63/429,534, filed on Dec. 1, 2022.

(51) Int. Cl. A61F 5/44; A61F 5/441; A61F 5/451

(52) U.S. Cl. CPC A61F 5/4405; A61F 5/441; A61F 5/451

(57) ABSTRACT

A dual use urine collection assembly comprising a drainage tube assembly having a drainage tube connected to a proximal chamber and an atmospheric equilibrium valve disposed in fluid communication with the proximal chamber. The atmospheric equilibrium valve has an airway with a cross-sectional air inlet area, and the airway is connected to the proximal chamber at an interface having a membrane area. The membrane area is in the range of about 4 to about 8 times larger than the air inlet area. The proximal chamber has a hydrophobic breathable membrane disposed on an inside surface of the proximal chamber over the interface.

### CROSS-REFERENCE TO RELATED APPLICATION

This application claims the benefit of U.S. Provisional Patent Application No. 63/429,534, filed on Dec. 1, 2022, the entire contents of which are hereby incorporated by this reference.

### BACKGROUND

### (1) Field of Endeavor

The device disclosed herein relates generally to the field of incontinence devices, and more particularly to an external, wearable incontinence device that may be used with a wearable collection assembly or as an external catheter connected to hospital wall suction units. The improved external incontinence devices disclosed herein are related to the drainage tube assembly of U.S. Pat. No. 10,022,261, the entire contents of which are incorporated herein by reference.

### (2) Description of Related Art

There is a need for an external wearable incontinence device that is compatible with both gravitational fluid flow and hospital wall suction (mechanical vacuum) units. Past external wearable incontinence devices have been difficult to attach. Many such devices incorporate skin adhesives to promote a consistently reliable liquid-tight seal that enables proper operation of the device. Example of such adhesives and seals are shown and described in U.S. Patent Application Publication No. 2013/0213415 and U.S. Pat. No. 8,551,062, respectively, the entire contents of each of which are incorporated herein by this reference. However, to avoid damaging skin tissue of the patient, many such adhesives should not be repeatedly applied and removed. These devices become unworkable in a hospital setting where patients may be ambulatory for periods of time, and then may be recovering in a bed for other periods of time. Ambulatory movement requires an external wearable incontinence device to properly operate under gravitational fluid flow, such as the device shown and described in U.S. Pat. No. 7,896,857, the entire contents of which are incorporated herein by this reference. Many such devices incorporate an atmospheric equilibrium valve, such as the one shown in U.S. Pat. No. 10,022,261. These valves are configured to alleviate pressure differences caused by gravitational fluid flow, and such pressure differences tend to be very small.

However, such gravity-driven atmospheric equilibrium valves are not compatible with the relatively high pressure differences caused by hospital wall suction units that are typically used to evacuate urine from incontinent patients who are recovering in a hospital bed. Thus, many incontinent hospital patients are faced with the dilemma of using improper external wearable devices, or repeatedly changing from a gravity-driven device to a wall suction device, thereby risking skin irritation and damage caused by repeatedly applying and removing skin adhesives.

Wall suction is advantageous to hospital patients in the intensive care unit when using an external incontinence catheter since it eliminates the need for a condom catheter or internal (Foley type) catheter and therefore reduces the well-known risk of catheter acquired urinary tract infection. Additionally, high flow rates improve the ability of the device to drawn fluids away from the patient at lower regulated vacuum settings.

Therefore, there is a need for an external wearable incontinence device that maximizes airflow while reducing fluid flow restrictions, thereby alleviating pressure differences caused by both gravity-driven fluid flow and by hospital wall suction units.

### SUMMARY

The device disclosed herein relates generally to the field of incontinence devices, and more particularly to an external, wearable incontinence device that may be used with a wearable collection assembly or as an external catheter connected to hospital wall suction units. The drainage tube assembly generally comprises a proximal chamber disposed in fluid communication with a drainage tube. An atmospheric equilibrium valve assembly is disposed on the exterior of the proximal chamber in fluid communication with the proximal chamber. The atmospheric equilibrium valve assembly may include one or more of a duckbill valve, an umbrella valve, a ball check valve, a push-pull valve, a twist push valve, a bridge check valve, or a sintered filter.

The atmospheric equilibrium valve assembly may also include a hydrophobic, breathable membrane affixed to an inner wall of the proximal chamber over an interface between the proximal chamber and an airway that opens to ambient air. The membrane permits passage of air to alleviate pressure differentials across the interface, but resists the passage of fluid, such as urine, passing through the proximal chamber.

In an embodiment, the breathable membrane comprises one or more pores, where the pore size less than about 10 micrometers but greater than about 2 micrometers. In an embodiment, the breathable membrane has a water breakthrough pressure in the range of about 1 psi to about 3 psi. In an embodiment, the breathable membrane enables airflow across the membrane in the range of about 80 standard liters per minute (SLM) to about 100 SLM. In an embodiment, the breathable membrane comprises surface modification, such as a fluoropolymer application, to promote wetting resistance against fluids flowing through the proximal chamber. In an embodiment, the airway has an open area of at least about 0.1 to about 0.4 square inches.

The bottom end of the drainage tube by comprise an anti-reflux valve, or it may be open ended. The anti-reflux valve may be preferable for embodiments that are predominantly used under gravitational flow in devices that incorporate an external wearable incontinence collection bag. In these embodiments, the anti-reflux valve prevents urine collected in the collection bag from entering into the drainage tube, which could risk the patient's health. The open end without an anti-reflux valve may be better suited for embodiments that are intended to be used predominantly with hospital wall suction units.

### BRIEF DESCRIPTION OF THE DRAWINGS

FIG. 1 shows a side view of one embodiment of a drainage tube assembly of the external incontinence device having an improved atmospheric equilibrium valve assembly that includes a perpendicular duck bill valve with breathable membrane.

FIG. 2 is another side view thereof.

FIG. 3 is an isometric view thereof.

FIG. 4 is a partial cross section view of the proximal chamber of the drainage tube assemble of the external incontinence device showing the atmospheric equilibrium valve assembly including a perpendicular duck bill valve with breathable membrane.

FIG. 5 shows a side view of another embodiment of a drainage tube assembly of the external incontinence device having an improved atmospheric equilibrium valve assembly that includes an angled duck bill valve with breathable membrane.

FIG. 6 is another side view thereof.

FIG. 7 is an isometric view thereof.

FIG. 8 is a partial cross section view of the proximal chamber of the drainage tube assemble of the external incontinence device showing the atmospheric equilibrium valve assembly including an angled duck bill valve with breathable membrane.

FIG. 9 shows a side view of another embodiment of a drainage tube assembly of the external incontinence device having an improved atmospheric equilibrium valve assembly that includes a parallel duck bill valve with breathable membrane.

FIG. 10 is another side view thereof.

FIG. 11 is an isometric view thereof.

FIG. 12 is a partial cross section view of the proximal chamber of the drainage tube assemble of the external incontinence device showing the atmospheric equilibrium valve assembly including a parallel duck bill valve with breathable membrane.

FIG. 13 shows a side view of another embodiment of a drainage tube assembly of the external incontinence device having an improved atmospheric equilibrium valve assembly that includes an umbrella valve.

FIG. 14 is another side view thereof.

FIG. 15 is an isometric view thereof.

FIG. 16 is a partial cross section view of the proximal chamber of the drainage tube assemble of the external incontinence device showing the atmospheric equilibrium valve assembly including an umbrella valve.

FIG. 17 is a cross section view of the umbrella valve of the embodiment shown in FIG. 16.

FIG. 18 shows a side view of another embodiment of a drainage tube assembly of the external incontinence device having an improved atmospheric equilibrium valve assembly that includes a ball check valve.

FIG. 19 is another side view thereof.

FIG. 20 is an isometric view thereof.

FIG. 21 is a partial cross section view of the proximal chamber of the drainage tube assemble of the external incontinence device showing the atmospheric equilibrium valve assembly including a ball check valve.

FIG. 22 is a cross section view of the ball check valve shown in FIG. 21.

FIG. 23 shows a side view of another embodiment of a drainage tube assembly of the external incontinence device having an improved atmospheric equilibrium valve assembly that includes a push-pull valve.

FIG. 24 is another side view thereof.

FIG. 25 is an isometric view thereof.

FIG. 26 is a partial cross section view of the proximal chamber of the drainage tube assemble of the external incontinence device showing the atmospheric equilibrium valve assembly including a push-pull valve.

FIG. 27 is a partial cutaway view of the push-pull valve in a closed position shown in FIG. 26.

FIG. 28 is a partial cutaway view of the push-pull valve in an open position shown in FIG. 26.

FIG. 29 is another partial cutaway view of the push-pull valve in a closed position shown in FIG. 26.

FIG. 30 shows a side view of another embodiment of a drainage tube assembly of the external incontinence device having an improved atmospheric equilibrium valve assembly that includes a twist push valve.

FIG. 31 is another side view thereof.

FIG. 32 is an isometric view thereof.

FIG. 33 is a partial cross section view of the proximal chamber of the drainage tube assemble of the external incontinence device showing the atmospheric equilibrium valve assembly including the twist push valve.

FIG. 34 is a side view of the twist push valve shown in FIG. 33.

FIG. 35 is a partial cutaway view of the twist push valve in an open position shown in FIG. 33.

FIG. 36 is a partial cutaway view of the twist push valve in a closed position shown in FIG. 33.

FIG. 37 shows a side view of another embodiment of a drainage tube assembly of the external incontinence device having an improved atmospheric equilibrium valve assembly that includes a bridge check valve.

FIG. 38 is another side view thereof.

FIG. 39 is an isometric view thereof.

FIG. 40 is a partial cross section view of the proximal chamber of the drainage tube assemble of the external incontinence device showing the atmospheric equilibrium valve assembly including the bridge check valve in a closed position.

FIG. 41 is a side view of the bridge check valve showing the flexible strip flush with the slot.

FIG. 42 is a partial cross section view of the proximal chamber of the drainage tube assemble of the external incontinence device showing the atmospheric equilibrium valve assembly including the bridge check valve in an open position allowing air in from atmosphere.

FIG. 43 is a side view of the bridge check valve showing the flexible strip raised away from the slot.

FIG. 44 is a perspective view of another embodiment of the drainage tube assembly of an external incontinence device having a sintered filter.

FIG. 45 is another perspective view thereof showing the sintered filter separated from the drainage tube assembly.

FIG. 46 illustrates another embodiment of the drainage tube assembly of an external male catheter having a hydrophobic membrane and configured to be compatible with suction.

FIG. 47 is cross-sectional view thereof showing the membrane secured within the proximal chamber of the drainage tube assembly.

FIG. 48 is a bottom perspective view of the embodiment shown in FIG. 46.

FIG. 49 is top view of the embodiment shown in FIG. 46.

FIG. 50 is a bottom view of the embodiment shown in FIG. 46.

FIG. 51 is a perspective view showing the embodiment of FIG. 46 attached to a parameatal seal at its top end and an adapter at its bottom end.

FIG. 52 is a perspective view showing the embodiment of FIG. 46 attached to a parameatal seal at its top end having an adapter detached at its bottom end.

FIG. 53 is a perspective view of the airway volume, showing the air inlet area in relation to the interface membrane area.

FIG. 54 is a perspective view of the embodiment of FIG. 46 attached to an embodiment of an external wearable collection bag.

FIG. 55 is a perspective view of the embodiment of FIG. 46 attached to an embodiment of an external wearable collection bag having a drainage conduit disposed therein.

### DETAILED DESCRIPTION OF THE PREFERRED EMBODIMENTS

With reference to the drawings, the dual use drainage tube assembly will now be described with regard for the best mode and the preferred embodiments. The embodiments disclosed herein are meant for illustration and not limitation of the invention. An ordinary practitioner will appreciate that it is possible to create many variations of the following embodiments without undue experimentation.

The embodiments described herein present the drainage tube assembly 5 generally in terms of a drainage tube 10 having a proximal chamber 25 with at least one atmospheric equilibrium valve 11 that actuates the opening and closing of an airway, which promoted fluid communication between the inside of the proximal chamber 25 and the external ambient air. In operation, a pressure differential is created across the atmospheric equilibrium valve 11 when fluid flows through the drainage tube assembly 5. The fluid flow causes the pressure inside the proximal chamber 25 to fall below the external pressure outside the drainage tube assembly 5, thereby creating the pressure differential across the valve 11. This pressure differential will arise under gravitational fluid flow or under fluid flow caused by a hospital wall suction unit. However, the pressure differential caused by gravitational flow is significantly lower than the pressure differential caused by a hospital wall suction unit.

In any of the embodiments discussed below, the atmospheric equilibrium valve 11 may or may not operate in connection with a breathable membrane disposed at an interface between the airway and the proximal chamber. When the breathable membrane is used, the interface defines a membrane area that is multiple times larger than an air inlet area defined by the cross sectional area of the airway. This ratio of membrane area to air inlet area enables air to flow across the breathable membrane without the breathable membrane restricting the airflow between the proximal chamber and the external ambient air.

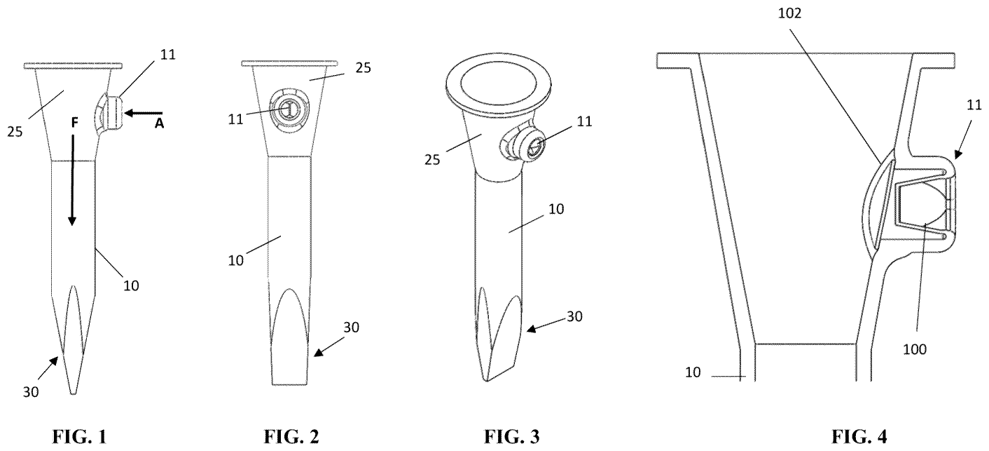

Referring to FIGS. 1 through 4, one embodiment of the dual use drainage tube assembly 5 is shown. The drainage tube assembly 5 comprises a proximal chamber 25 disposed in fluid communication with a drainage tube 10, the proximal chamber 25 having a frustoconical shape. The drainage tube assembly 5 may further include an anti-reflux valve 30. In this embodiment, the atmospheric equilibrium valve assembly 11 is disposed on the exterior of the proximal chamber 25 and is disposed in fluid communication with the proximal chamber 25. The atmospheric equilibrium valve assembly 11 has a built-in duckbill valve 100 disposed in an orientation where the air passing through an airway defined by the valve, indicated by arrow A in FIG. 1, flows in a direction substantially perpendicular to the direction of fluid flowing through the drainage tube assembly 5, indicated by arrow F in FIG. 1. The duck bill valve 100 enables air to enter when the pressure differential arises.

The atmospheric equilibrium valve assembly 11 may also include a breathable membrane 102 affixed to an inner wall of the proximal chamber 25 and over an interface area where the airway defined by the built-in duckbill valve assembly is disposed in fluid communication with the proximal chamber 25. The breathable membrane 102, described in more detail below, resists fluids from reaching the duckbill valve 100 directly, reducing the risk of leakage.

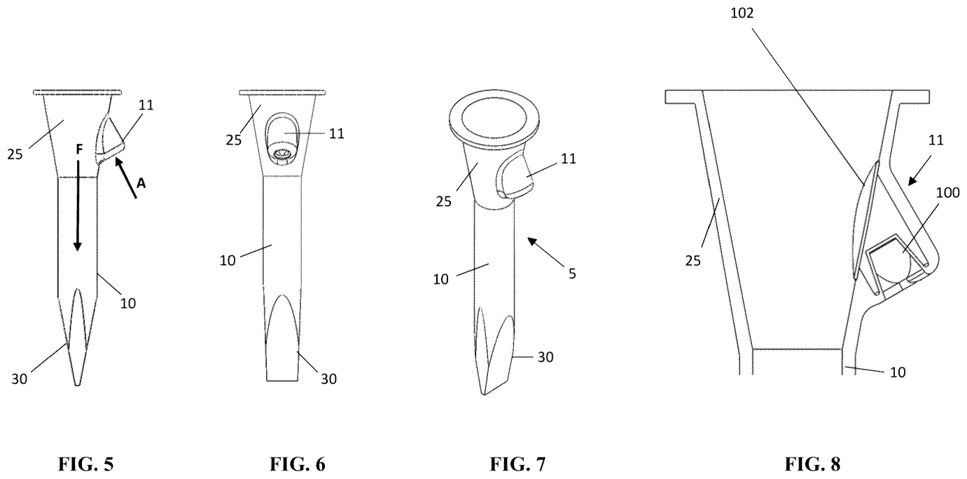

Referring to FIGS. 5 through 8, another embodiment of the drainage tube assembly 5 is shown. In this embodiment, the built-in duck bill valve 100 may be disposed in the side of the proximal chamber 25 where the direction of airflow A through the airway is at an angle in relation to the direction of fluid flow F. The angle between direction A and direction F is preferably in the range of about 15 degrees to about 60 degrees.

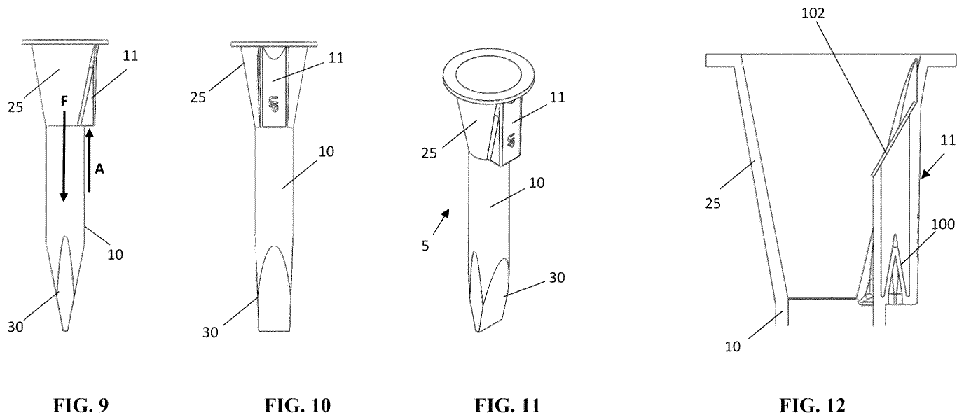

Referring to FIGS. 9 through 12, another embodiment of the drainage tube assembly 5 is shown. In this embodiment, the built-in duck bill valve 100 may be oriented such that airflow direction A is substantially parallel to the direction of fluid flow F in the proximal chamber 25.

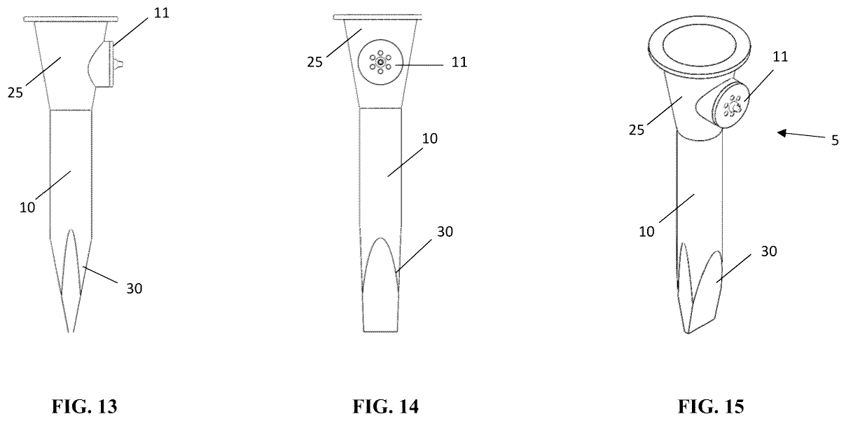

Referring to FIGS. 13 through 17, another embodiment of the drainage tube assembly 5 is shown. The drainage tube assembly 5 comprises a proximal chamber 25 disposed in fluid communication with a drainage tube 10, the proximal chamber 25 having a frustoconical shape. The drainage tube assembly 5 may further include an anti-reflux valve 30. In this embodiment, the atmospheric equilibrium valve assembly 11 is disposed on the exterior of the proximal chamber 25 and is disposed in fluid communication with the proximal chamber 25. The atmospheric equilibrium valve assembly 11 is an umbrella valve.

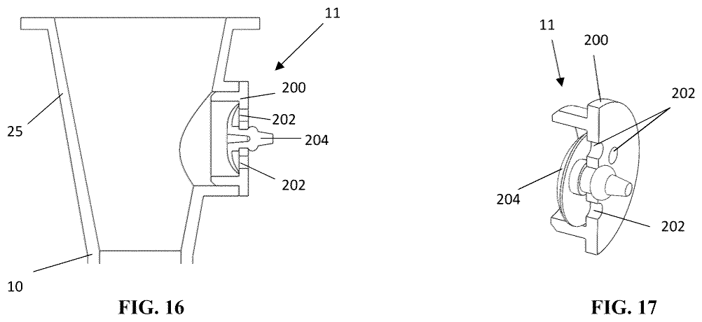

Referring to FIGS. 16 and 17, the umbrella valve 11 comprises a rigid cap 200, at least one hole 202, and a flexible umbrella 204. The rigid cap 200 is assembled by interface fit onto the proximal chamber 25. The cap 200 has a central perforation where the flexible umbrella 204 is secured. When a pressure differential arises in the drainage tube assembly 5, the umbrella 204 deforms, defining an airway that enables airflow into the proximal chamber 25. When the pressure differential dissipates, the umbrella 204 makes a seal with the cap to close the airway and prevent urine from leaking from the proximal chamber 25.

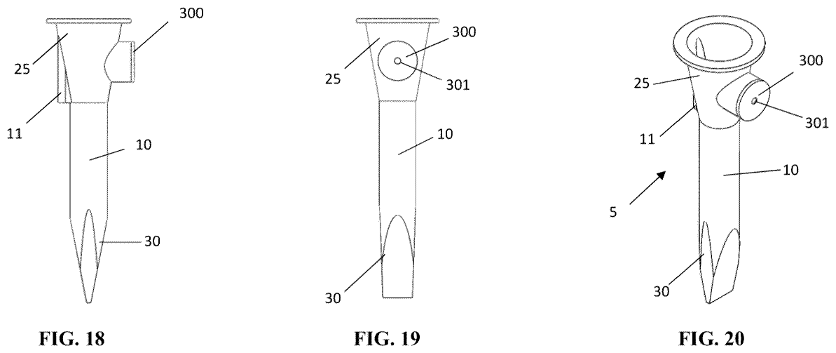

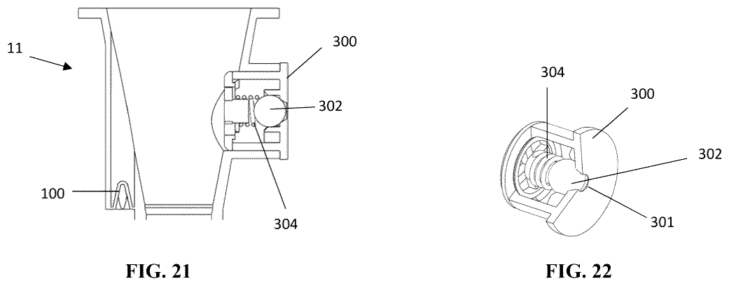

Referring to FIGS. 18 through 22, the drainage tube assembly 5 may include an atmospheric equilibrium valve 11 having a ball check valve 300. The ball check valve 300 is disposed on the proximal chamber 25 in fluid communication with the proximal chamber 25. The drainage tube assembly 5 with the ball check valve 300 may also include a duckbill vent valve 11 disposed on the proximal chamber 25 in fluid communication therewith. An outer cap 300 of the ball check valve has an air intake hole 301 in its center. It also has a central core section with a spring 304 which biases a ball 302 against the hole 301 to seal the ball check valve 300. When the pressure differential arises during operation of the drainage tube assembly 5, the external atmospheric pressure moves the ball 302 away from the hole 301, thereby compressing the spring 304 to open an airway so that external air can enter the proximal chamber 25. When the pressure differential dissipates, the spring 304 pushes the ball 302 against the hole 301 to close the airway and to prevent fluid leakage from the proximal chamber 25.

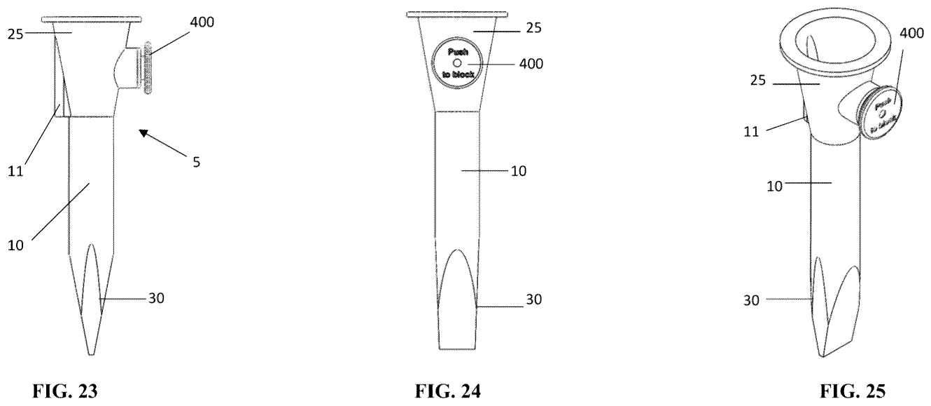

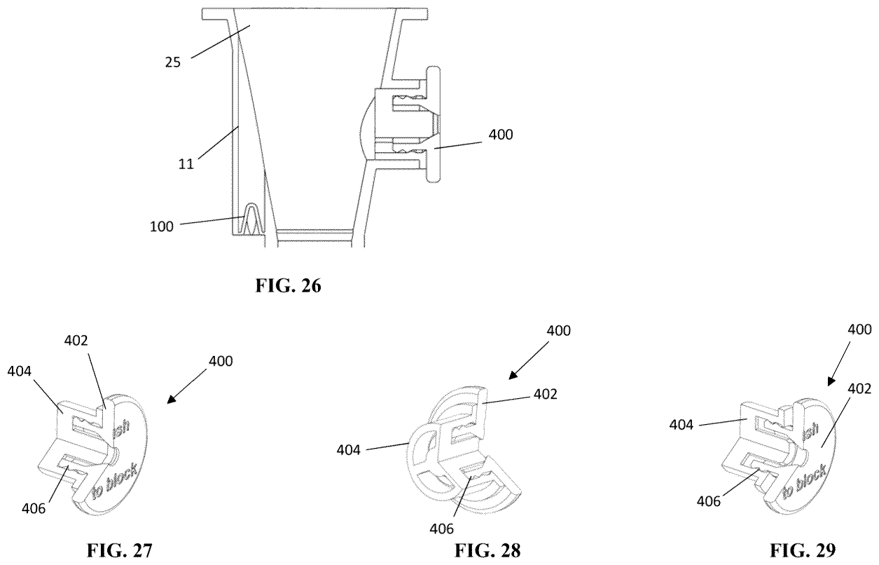

Referring to FIGS. 23 through 29, the drainage tube assembly 5 may include a push-pull valve assembly 400. The push-pull valve assembly 400 is disposed on the proximal chamber 25 in fluid communication with the proximal chamber 25. The drainage tube assembly 5 with the push-pull valve assembly 400 may also include a duckbill valve 11 disposed on the proximal chamber 25 in fluid communication therewith. The push-pull valve assembly 400 includes a cap 402, a base 404, and grooves 406. The cap 402 is coupled to the base 404. Both the cap 402 and the base 404 have corresponding grooves 406 that enable them to retain their position when the cap 402 is pushed or pulled. When the pressure differential arises, the user must pull the push-pull cap 402, causing separation between the cap 402 and the base 404 to open an airway. This enables airflow through the airway so that the liquid contents of the proximal chamber 25 can be evacuated. When the evacuation process is complete, the user should press the cap 402 to block the flow and prevent any leakage.

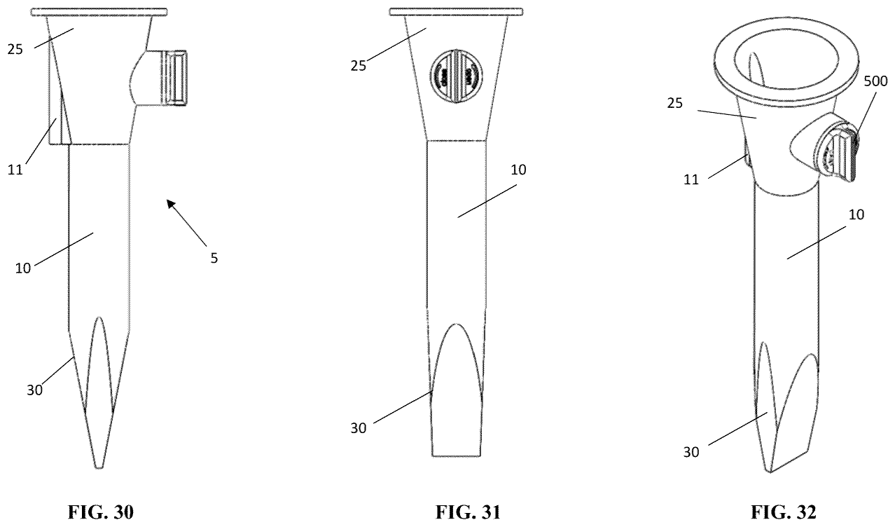

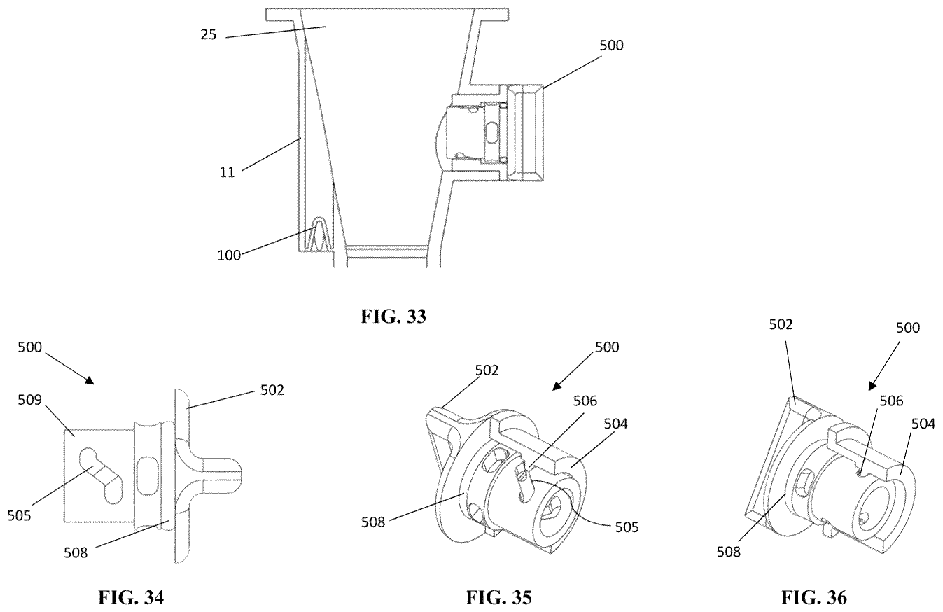

Referring to FIGS. 30 through 36, the drainage tube assembly 5 may include a twist push valve assembly 500. The twist push valve assembly 500 is disposed on the proximal chamber 25 in fluid communication with the proximal chamber 25. The drainage tube assembly 5 with the twist push valve assembly 500 may also include a duckbill valve 11 disposed on the proximal chamber 25 in fluid communication therewith. The twist push valve assembly 500 includes a knob cap 502, a base 504, a slot 505, and a protrusion 506, and an o-ring 508. The knob cap 502 is coupled to the base 504. The slot 505 is located on the knob cap 502 and mates with the protrusion 506 on the base 504 such that the protrusion 506 is slidingly disposed in the slot 505. The slot 505 is shaped so that when the knob cap 502 is twisted, it moves relative to the base 504 in an axial direction, thereby separating the base 504 from the o-ring 508 and opening an airway that enables air into the proximal chamber 25. When the pressure differential arises during operation of the drainage tube assembly 5, the user must turn the knob cap 502, which causes separation between the o-ring 508 and the base 504. This enables air to enter through slots in the knob cap 502 to promote evacuation of urine from the drainage tube assembly 5. Once the evacuation process is complete and the pressure differential dissipates, the knob cap 502 should be turned back to its original position, which returns the o-ring 508 into contact with the base 504, closing the airway and preventing further airflow. In a variation of this embodiment, the slot 505 is disposed on the base 504, and the mating protrusion 506 is disposed on the knob cap 502.

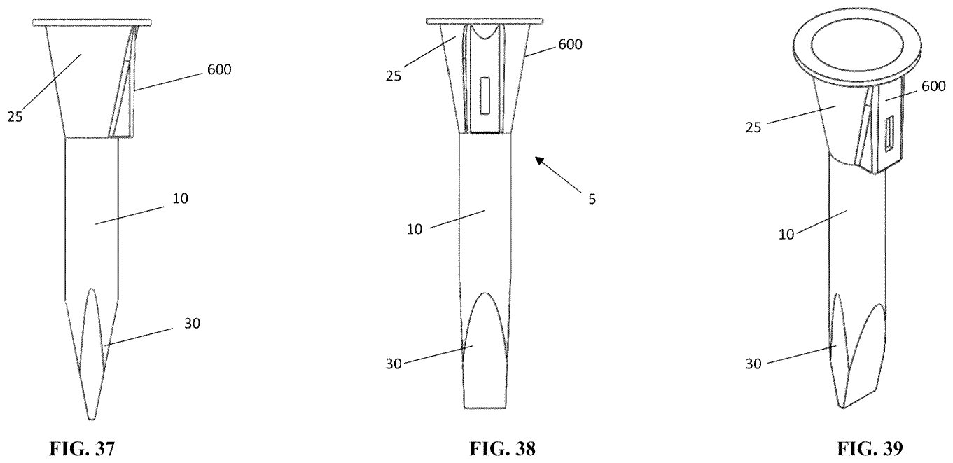

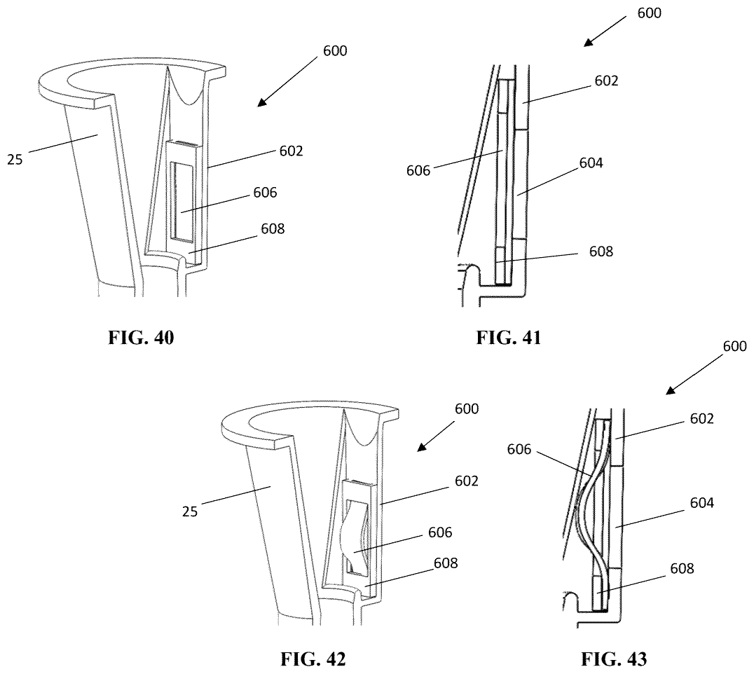

Referring to FIGS. 37 through 43, another embodiment of the drainage tube assembly 5 may include a bridge check valve assembly 600 disposed in fluid communication with the proximal chamber 25. The drainage tube assembly 5 with the bridge check valve assembly 600 may also include a duckbill valve 11 disposed on the proximal chamber 25 in fluid communication therewith. The bridge check valve assembly 600 includes a flat face 602, a slot 604, a flexible strip 606, and a frame 608. The slot 604 is located on the flat face 602 and the flexible strip 606 is secured by the frame 608 at two ends whereby the flexible strip 606 is held over and flush with the slot 604. When the pressure inside the bag is greater than or equal to the pressure outside the bag, the flexible strip 606 is pushed against the slot 604, which is smaller, thus creating a seal that prevents leakage. When fluid passes through the drainage tube assembly 5 to cause the pressure differential, the external pressure pushes and deforms the flexible strip 606 through the frame 608, thus opening the slot 604 to define an airway that enables air to flow into the proximal chamber 25.

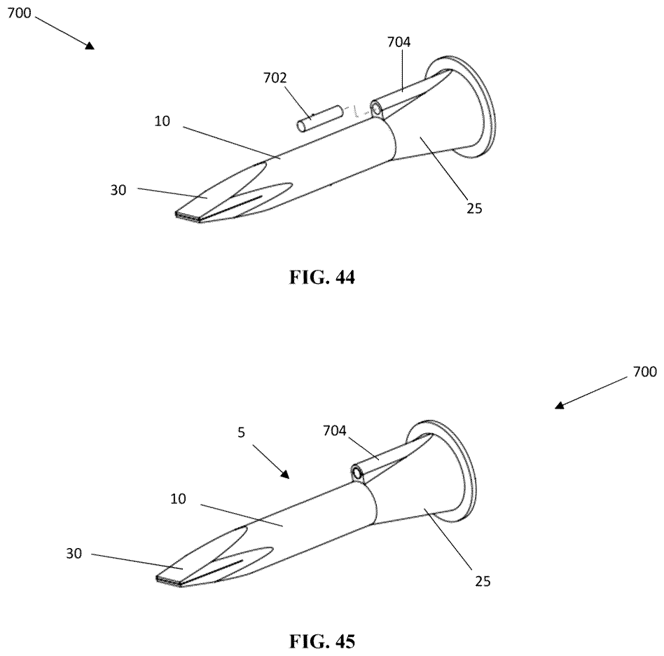

Referring to FIGS. 44 and 45, another embodiment of the external male catheter 700 may include a sintered filter 702 which may be secured into an equilibrium opening 704 located on the proximal chamber 25. The sintered filter 702 may be configured to resist deformation in the presences of the pressure differential, thereby reducing the risk of harm to the patient.

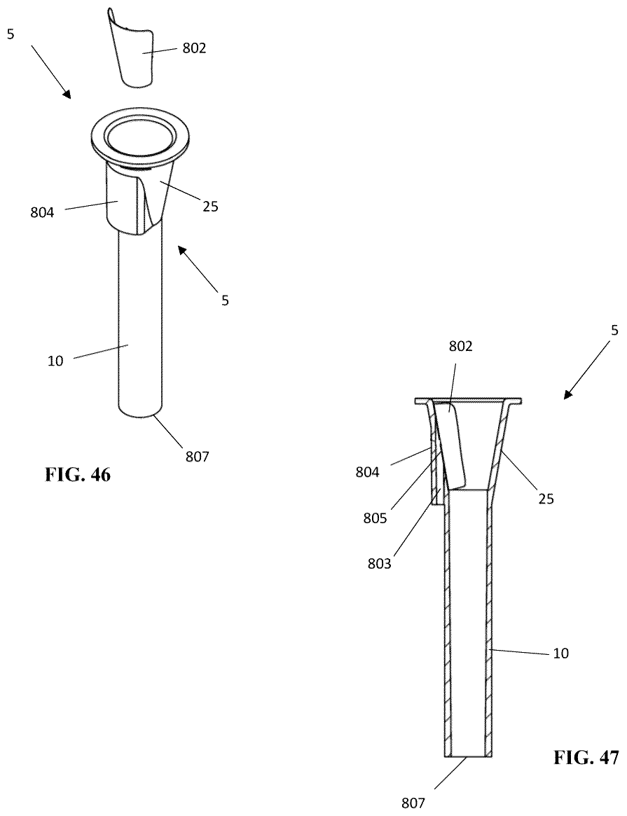

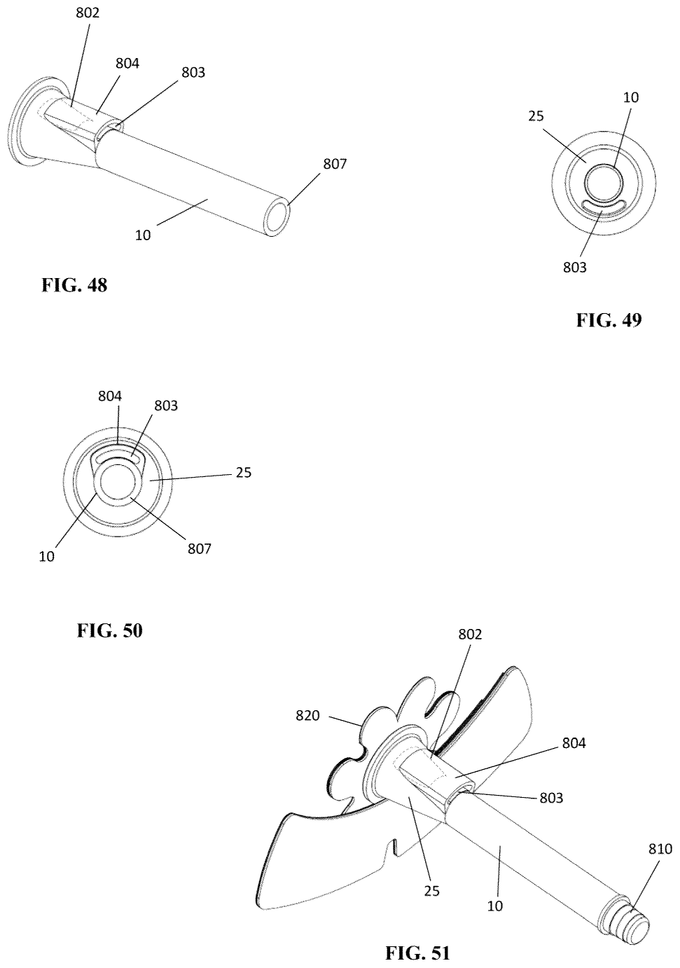

Referring to the embodiment shown in FIGS. 46-52, a proximal chamber 25 of a drainage tube assembly 5 is shown with a breathable membrane 802. In this embodiment, the drainage tube assembly 5 comprises an airway vent 804 having an airway 803 disposed on the proximal chamber 25 at an orientation where the direction of flow of air through the airway 803 is substantially parallel to the direction of flow of the fluid passing through the proximal chamber 25 (A and F in FIG. 9). A top portion of the airway 803 connects to the proximal chamber at an interface 805. A breathable membrane 802 is disposed on the inside surface of the proximal chamber 25 in a manner that covers the interface 805. The breathable membrane 802 enables airflow in the presence of the pressure differential while resisting water egress, thereby permitting the passage of air without permitting passage of the urine or other liquid flowing through the proximal chamber 25. The breathable membrane 802 is attached to the inside surface of the proximal chamber 25 by any suitable connection or bond, such as by an adhesive, an RF weld, an ultrasonic weld, a heat weld, or a suitable mechanical attachment.

Since the breathable membrane 802 attaches to the tapered interior surface of the proximal chamber 25, the three-dimensional nature of the frustoconical surface results in a larger membrane 802 surface area than if the membrane 802 were attached to a flat surface. The same size opening on a two-dimensional surface would reduce the surface area of the membrane 802 by about 12%.

The airway vent 804 on the proximal chamber 25 enables ambient air to enter through the airway 803 when the drainage tube assembly 5 is disposed in communication with hospital wall suction. In one embodiment of the airway vent 804, the airway 803 is disposed in a contour that matches the outside surface of the proximal chamber 25. Thus, the airway 803 is curved, or “kidney shaped.” Referring to FIG. 53, the cross sectional area of the kidney shaped opening defines an air inlet area that functions as the flow area through the airway. This area is less than the membrane area of the interface 805, which is frustoconical in shape as described above. Since the air inlet area is smaller than the membrane area, the flow rate and pressure in the airway 803 will be greater than the flow rate and pressure across the interface 805. For example, in an embodiment, the air inlet area is in the range of about 0.015 square inches to about 0.05 square inches, and the membrane area is in the range of about 0.125 square inches to about 0.2 square inches. Preferably, the air inlet area is in the range of about 0.025 square inches to about 0.030 square inches, and the membrane area is in the range of about 0.155 square inches to about 0.165 square inches. The membrane area is in the range of about 4 to about 8 times larger than the air inlet area, and preferably in the range of about 5.5 to about 6.5 times larger than the air inlet area. Disposing the membrane area and the air inlet area in these ranges enables the drainage tube assembly 5 to operate under both gravitational fluid flow and under hospital wall suction without the breathable membrane 802 restricting air flow across the interface 805.

Based on the foregoing ranges of membrane areas and air inlet areas, this embodiment of the drainage tube assembly 5 can be operated at a hospital wall suction pressure in the range of about 40 to about 80 mmHg, and up to a maximum of about 120 mmHg. High flow rates through the breathable membrane 802 and a sealed drainage tube assembly 5 enable the this embodiment of the drainage tube assembly 5 to perform at lower vacuum rates than prior art devices. This embodiment of the drainage tube assembly 5 operates at vacuum as low as about 40 mmHg and does not need to be set higher than about 80 mmHg to accommodate large flow rates from the patient. In competitive benchtop testing, the present drainage tube assembly 5 evacuated fluid 2 to 2.5 times faster (500 mL of fluid, 21 mL/sec fluid flow rate, 80 mmHg) that comparable prior art devices. High flow rates through the breathable membrane 802 minimize the risk to patient safety. Vacuum testing demonstrated an average pressure exerted on the glans of the male anatomy was about 30 mmHg when the regulated wall suction was set to about 160 mmHg (twice the recommended setting). For comparison, wound vacuums are typically set to about 120 mmHg and do not damage surrounding tissue.

The breathable membrane 802 is made of a porous material to enable compatibility with both gravitational fluid flow and with a wall suction system that can be connected to the bottom end 807 of the drainage tube assembly 5. For example, in one embodiment, the breathable membrane 802 is made of a hydrophobic or oleophobic material, such as a non-woven acrylic polymer. Alternately, the breathable membrane 802 could be a hydrophobic polymer membrane.

In an embodiment, the breathable membrane 802 comprises one or more pores, where the pore size less than about 10 micrometers but greater than about 2 micrometers. In an embodiment, the breathable membrane 802 has a water breakthrough pressure in the range of about 1 psi to about 3 psi. In an embodiment, the breathable membrane 802 enables airflow across the membrane 802 in the range of about 80 standard liters per minute (SLM) to about 100 SLM, and preferably in the range of about 85 SLM to about 91 SLM. In an embodiment, the breathable membrane 802 comprises surface modification, such as a fluoropolymer application, to promote wetting resistance against fluids flowing through the proximal chamber 25. In an embodiment, the airway 803 has an open area of at least about 0.1 to about 0.4 square inches. In an embodiment, the airway 803 has an opening of about 0.233 square inches.

In some embodiments, multiple breathable membranes 802 could be used, having various properties as discussed herein, to adjust the flow rate, airflow, pressure, or breathability of the overall system. In this embodiment, the multiple breathable membranes 802 may be layered over the interface 805. For example, a first breathable membrane 802 layer may have one or more pores, where the pore size less than about 10 micrometers but greater than about 2 micrometers. A second breathable membrane 802 layer may have a water breakthrough pressure in the range of about 1 psi to about 3 psi. A third layer may enable airflow across the membrane 802 in the range of about 80 standard liters per minute (SLM) to about 100 SLM, and preferably about 85 SLM to about 91 SLM. A fourth layer may comprise surface modification, such as a fluoropolymer application, to promote wetting resistance against fluids flowing through the proximal chamber 25. These membranes 802 may be layered in any order or in any combination to achieve the desired performance of the breathable membrane 802 in the drainage tube assembly 5. In any of the embodiments discussed above, the breathable membrane 802 could be used in connection with any of the disclosed embodiments of the atmospheric equilibrium valve 11.

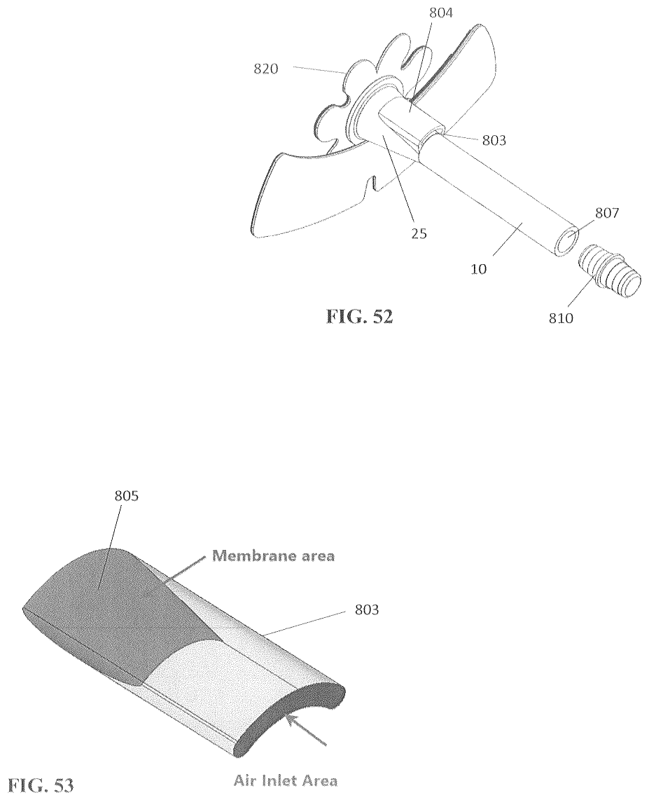

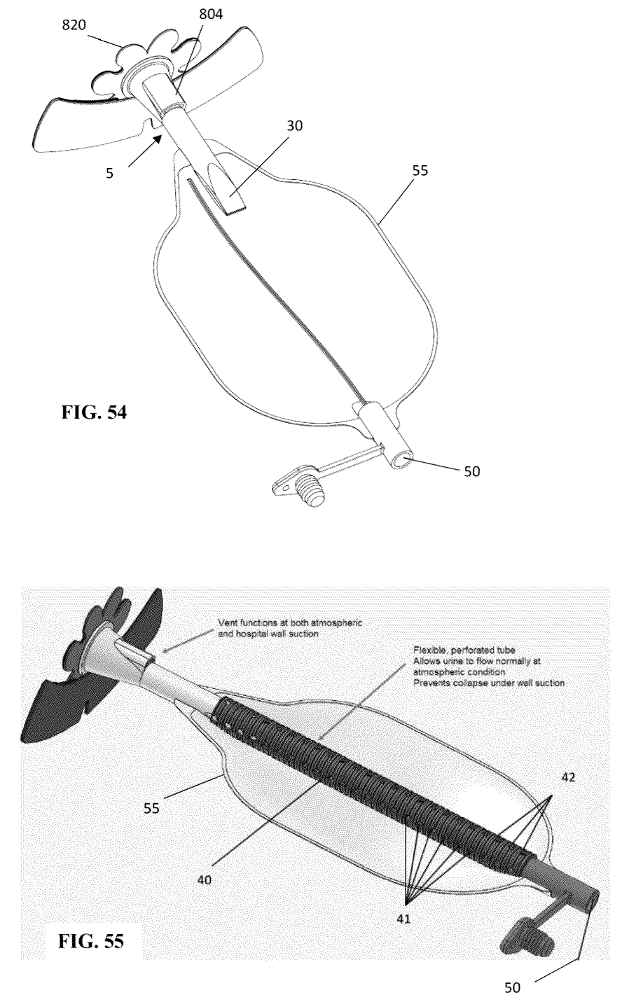

Some prior art systems are not completely sealed, thus risking leakage. Referring to FIGS. 51 and 52, embodiments of the present drainage tube assembly 5 may be attached to a parameatal seal 820 for attachment to the user, as discussed in U.S. Pat. No. 8,551,062. In these embodiments, the entire system for the drainage tube assembly 5 becomes a sealed system from the parameatal seal, through the drainage tube assembly 5, and through the external collection bag 55 (see FIGS. 54 and 55) or the hospital wall suction. Thus, these embodiments rely on the atmospheric equilibrium valve 11 for proper functionality of the drainage tube assembly 5. Without the atmospheric equilibrium valve 11, the urine will not flow properly through the sealed system. The atmospheric equilibrium valves 11 described herein provide the unexpected result of enabling the drainage tube assembly 5 to function properly under the two very different states of pressure differential caused by gravitational fluid flow on the one hand, and by hospital wall suction on the other hand.

In some uses, the drainage tube assembly 5 may be attached to either an external collection bag or to hospital wall suction by either male or female connectors on the mating device. For example, hospital vacuum tubing typically uses a flexible female connector, while bed and leg urine collection bags have a rigid, stepped, male connector, often comprising a hose barb. An embodiment of the present drainage tube assembly 5 comprises a removable adapter 810 to accommodate flexible vacuum tubing and can be removed to accommodate rigid male connectors. The bore of the adapter is large enough to prevent a reduction in fluid flow rates. Thus, any of the embodiments described above may be fitted with an adapter 810 that is suitable for making male to male connections or female to female connections. To attach the adapter 810, the bottom end 807 of the drainage tube assembly 5 functions as a female connector that receives the male hose barb insert of the adapter 810.

In any of the foregoing embodiments, the bottom end 807 of the drainage tube 10 may comprise an anti-reflux valve 30 as shown in FIGS. 1-3, or it may be open as shown in FIGS. 46 and 47. The anti-reflux valve 30 may be preferable for embodiments that are predominantly used under gravitational flow in devices that incorporate an external wearable incontinence collection bag 55. In these embodiments, the anti-reflux valve 30 prevents urine collected in the collection bag 55 from entering into the drainage tube 10, which could risk the patient's health. The open end shown in FIGS. 46 and 47 may be better suited for embodiments that are intended to be used predominantly with hospital wall suction units.

Referring to FIG. 55, one embodiment of a suitable external wearable urine collection bag 55 comprises a drainage conduit 40 that provides structural support to the collection bag 55, particularly when the collection bag 55 is placed under the vacuum of a wall suction unit in a hospital or other healthcare setting. Wall suction units provide significantly greater suction force than that caused by gravitational forces under atmospheric pressure. This greater suction force tends to collapse the urine collection bag 55, often disrupting flow through the bag and causing great discomfort to the user.

The drainage conduit 40 comprises a plurality of apertures 41 for enabling passage of urine into and out of the collection bag 55, and for passing the urine into the drainage conduit 40 when used as a system dependent on gravity. Collected urine can then be removed from the collection bag 55 through the evacuation port 50. Urine can flow directly through the drainage conduit 40 to the evacuation port 50 when a vacuum source is applied to the evacuation port 50.

The apertures 41 are configured as slots or as holes having a circular, polygonal, or other suitable shape. The drainage conduit 40 further comprises a plurality of annular reinforcing elements 42, such as annular ribs or ridges disposed in a spiral-like or ring-like orientation about the drainage conduit 40 to increase the hoop strength of the drainage conduit 40. In one embodiment, the drainage conduit 40 is constructed of flexible, resilient material to promote bending of the conduit 40. However, the reinforcing elements 42 enable the conduit 40 to retain its shape under the suction force of wall suction, thereby providing structural support to the collection bag 1 to prevent undesirable collapse.

The foregoing embodiments are merely representative of the dual use drainage tube assembly and not meant for limitation of the invention. For example, persons skilled in the art would readily appreciate that there are several embodiments and configurations of valves, breathable membranes, and other components of the drainage tube assembly that will not substantially alter the nature of the present device. Likewise, elements and features of the disclosed embodiments could be substituted or interchanged with elements and features of other embodiments, as will be appreciated by an ordinary practitioner. Consequently, it is understood that equivalents and substitutions for certain elements and components set forth above are part of the invention described herein, and the true scope of the invention is set forth in the claims below.

What is claimed is:

1. A drainage tube assembly for an external incontinence device, the drainage tube assembly comprising:
a drainage tube connected to a proximal chamber; and
an atmospheric equilibrium valve disposed in fluid communication with the proximal chamber, the atmospheric equilibrium valve having an airway with a cross-sectional air inlet area, the airway connecting to the proximal chamber at an interface having a membrane area, the membrane area being in the range of about 4 to about 8 times larger than the air inlet area; and
a hydrophobic breathable membrane disposed on an inside surface of the proximal chamber over the interface.

2. The drainage tube assembly for an external incontinence device of claim 1, wherein the drainage tube comprises a bottom end, and the drainage tube assembly further comprises an adapter for connecting the bottom end either to a flexible female connector or to a stepped male connector.

3. The drainage tube assembly for an external incontinence device of claim 1, wherein the breathable membrane comprises pores having a size in the range of about 5 micrometers to about 10 micrometers and a water breakthrough pressure in the range of at least about 1 psi to about 3 psi, and the breathable membrane enables airflow across the breathable membrane in the range of about 80 standard liters per minute (SLM) to about 100 SLM.

4. The drainage tube assembly for an external incontinence device of claim 1, wherein the breathable membrane comprises multiple layers, a first layer having one or more pores, where the pore size less than about 10 micrometers but greater than about 2 micrometers, a second layer having a water breakthrough pressure in the range of about 1 psi to about 3 psi, and a third layer that enables airflow across the breathable membrane in the range of about 80 standard liters per minute (SLM) to about 100 SLM.

5. The drainage tube assembly for an external incontinence device of claim 1, further comprising an external wearable collection bag having a drainage conduit disposed therein.

6. The drainage tube assembly for an external incontinence device of claim 1, wherein the atmospheric equilibrium valve comprises one or more of a duckbill valve, an umbrella valve, a ball check valve, a push-pull valve, a twist push valve, a bridge check valve, or a sintered filter.

7. The drainage tube assembly for an external incontinence device of claim 2, wherein the breathable membrane comprises pores having a size in the range of about 5 micrometers to about 10 micrometers and a water breakthrough pressure in the range of at least about 1 psi to about 3 psi, and the breathable membrane enables airflow across the breathable membrane in the range of about 80 standard liters per minute (SLM) to about 100 SLM.

8. The drainage tube assembly for an external incontinence device of claim 2, wherein the breathable membrane comprises multiple layers, a first layer having one or more pores, where the pore size less than about 10 micrometers but greater than about 2 micrometers, a second layer having a water breakthrough pressure in the range of about 1 psi to about 3 psi, and a third layer that enables airflow across the breathable membrane in the range of about 80 standard liters per minute (SLM) to about 100 SLM.

9. The drainage tube assembly for an external incontinence device of claim 3, further comprising an external wearable collection bag having a drainage conduit disposed therein.

10. The drainage tube assembly for an external incontinence device of claim 3, wherein the atmospheric equilibrium valve comprises one or more of a duckbill valve, an umbrella valve, a ball check valve, a push-pull valve, a twist push valve, a bridge check valve, or a sintered filter.

11. A drainage tube assembly for an external incontinence device, the drainage tube assembly comprising:
a drainage tube connected to a proximal chamber; and
two or more atmospheric equilibrium valves disposed in fluid communication with the proximal chamber, at least one of the two or more atmospheric equilibrium valves having an airway with a cross-sectional air inlet area, the airway connecting to the proximal chamber at an interface having a membrane area, the membrane area being in the range of about 4 to about 8 times larger than the air inlet area; and
a hydrophobic breathable membrane disposed on an inside surface of the proximal chamber over the interface.

12. The drainage tube assembly for an external incontinence device of claim 11, wherein the drainage tube comprises a bottom end, and the drainage tube assembly further comprises an adapter for connecting the bottom end either to a flexible female connector or to a stepped male connector.

13. The drainage tube assembly for an external incontinence device of claim 11, wherein the breathable membrane comprises pores having a size in the range of about 5 micrometers to about 10 micrometers and a water breakthrough pressure in the range of at least about 1 psi to about 3 psi, and the breathable membrane enables airflow across the breathable membrane in the range of about 80 standard liters per minute (SLM) to about 100 SLM.

14. The drainage tube assembly for an external incontinence device of claim 11, wherein the breathable membrane comprises multiple layers, a first layer having one or more pores, where the pore size less than about 10 micrometers but greater than about 2 micrometers, a second layer having a water breakthrough pressure in the range of about 1 psi to about 3 psi, and a third layer that enables airflow across the breathable membrane in the range of about 80 standard liters per minute (SLM) to about 100 SLM.

15. The drainage tube assembly for an external incontinence device of claim 11, further comprising an external wearable collection bag having a drainage conduit disposed therein.

16. The drainage tube assembly for an external incontinence device of claim 11, wherein the atmospheric equilibrium valve comprises one or more of a duckbill valve, an umbrella valve, a ball check valve, a push-pull valve, a twist push valve, a bridge check valve, or a sintered filter.

17. The drainage tube assembly for an external incontinence device of claim 12, wherein the breathable membrane comprises pores having a size in the range of about 5 micrometers to about 10 micrometers and a water breakthrough pressure in the range of at least about 1 psi to about 3 psi, and the breathable membrane enables airflow across the breathable membrane in the range of about 80 standard liters per minute (SLM) to about 100 SLM.

18. The drainage tube assembly for an external incontinence device of claim 12, wherein the breathable membrane comprises multiple layers, a first layer having one or more pores, where the pore size less than about 10 micrometers but greater than about 2 micrometers, a second layer having a water breakthrough pressure in the range of about 1 psi to about 3 psi, and a third layer that enables airflow across the breathable membrane in the range of about 80 standard liters per minute (SLM) to about 100 SLM.

19. The drainage tube assembly for an external incontinence device of claim 13, further comprising an external wearable collection bag having a drainage conduit disposed therein.

20. The drainage tube assembly for an external incontinence device of claim 13, wherein the atmospheric equilibrium valve comprises one or more of a duckbill valve, an umbrella valve, a ball check valve, a push-pull valve, a twist push valve, a bridge check valve, or a sintered filter.
